(19) United States
(12) Patent Application Publication
(10) Pub. No.: US 2019/0107250 A1
(43) Pub. Date: Apr. 11, 2019

(54) RESERVOIR WITH FRANGIBLE SEAL AND WICKING MATERIAL FOR THE LUBRICATION OF A SURGICAL DEVICE

(71) Applicant: Proximate Concepts LLC, Fort Lee, NJ (US)

(72) Inventor: Paul Rosenberg, Saddle River, NJ (US)

(21) Appl. No.: 15/726,768

(22) Filed: Oct. 6, 2017

(51) Int. Cl. F16N 7/12; B65B 3/04

(52) U.S. Cl. CPC F16N 7/12; A61B 90/08; B65B 3/04

(57) ABSTRACT

Systems and methods provided herein concern a fluid delivery system for use with medical devices and, more specifically, for activating surface coatings of medical devices prior to use in medical procedures. The fluid delivery system comprises one or more sheets of biocompatible wicking material that is attached to a reservoir. Sealed within the reservoir is a fluid, such as physiologic saline or other appropriate hydrating solution. In addition, the reservoir comprises at least one outlet, such as a frangible seal, that can be selectively breached prior to use by applying pressure to the reservoir, e.g., by squeezing or twisting the reservoir. Breaching the frangible seal thus releases the fluid onto the wicking material, which, in turn, serves to wet the medical device and consequently activate the surface coating.

### FIELD OF THE INVENTION

The invention relates to surgical devices and more specifically, a system for the delivery of fluids for use with surgical instruments that require lubrication, or the activation of a lubricant, before or during use.

### BACKGROUND

The invention relates to surgical devices for assisting in surgical procedures and, more specifically, a fluid delivery system for use with surgical devices that require lubrication, or the activation of a lubricant, before or during use.

Many medical devices, for example those which are implantable or otherwise intended for insertion within a body during a medical procedure, are coated with any number of surface treatments that are designed to perform various functions. These coatings are typically covalently bonded to the surface substrate of the medical device so that the surface coating performs a certain action when making contact with tissues inside the body or inside the lumen of an internal organ. This action might include resistance against blood clots or thrombus formation, resistance against infection, resistance against surface tension, etc. For example, such coatings can generally be broken down into four main categories, namely, hydrophilic coatings, hydrophobic coatings, antimicrobial coatings, anti-thrombotic coatings, although other types of coatings exist and are know to those of skill in the art.

Typically, such topical coatings are fused to the surface or substrate of the medical device, e.g., by heat curing or ultraviolet curing. This energy transfer activates a covalent bond between the coating substance and the substrate that receives the coating. Such topical coatings often need to be activated or, in other words, transferred from an inactive phase to an active phase. This activation is typically performed through the application of water or physiologic saline solution to the coated surface. More specifically, the typical activation process for a surface coating is that the device is first removed from a sterile pack in which it came from the manufacturer and it is then either submerged in a vessel containing sufficient water as to cover the device, or placed in a receptacle such that water is added to cover the device. A third method for activation is to open the sterile packaging and use a syringe to irrigate the device with solution, thereby activating the coating.

Problems with existing fluid delivery systems include inefficient use of time and materials in the operating room while preparing the medical device for use. Using current techniques, activation of a coating on a medical device requires submerging of the device within a volume of activating solution that exceeds the area requiring activation. For example, submerging the device within a basin of saline to activate a coating on a surface of the device. This also raises issue of sterilization when the device must contact multiple surfaces during activation of a coating and prior to use on a patient.

Accordingly, what is needed is a fluid delivery system that can be used to selectively and controllably apply sterilized fluids to a medical device, such as those devices that require wetting before or during use to activate a surface coating. What is further needed is a fluid delivery system in which the fluid delivery device and the fluid contained therein (and optionally the medical device with which it is intended to be used) can be sterilized without requiring separate sterilization of the device and the fluid for combination in a sterile environment.

It is with respect to these and other considerations that the disclosure made herein is presented.

### SUMMARY

According to one embodiment a fluid delivery system for use with a medical device is disclosed. The fluid delivery system comprises a reservoir that is formed of one or more exterior walls arranged to define an internal volume and configured to contain a fluid therein. The reservoir further comprises a frangible seal that is configured to rupture in response to the application of a sufficient amount of pressure on one or more of the frangible seal and the one or more exterior walls. Rupture of the frangible seal provides an outlet for the release of the fluid contained within the reservoir. The fluid delivery system also comprises a wicking material that is coupled or otherwise affixed to the reservoir. The wicking material is configured to absorb the volume of the fluid released from the reservoir, distributing the fluid across at least a portion of the wicking material.

Various features, aspects and advantages in accordance with embodiments of the invention can be appreciated from the following detailed descriptions.

### DESCRIPTION OF THE DRAWING FIGURES

The invention is illustrated in the figures of the accompanying drawings which are meant to be exemplary and not limiting, in which like references are intended to refer to like or corresponding parts, and in which:

FIG. 1A illustrates a top perspective view of a reservoir with a frangible seal and holding a sterilized fluid for the lubrication of a surgical device according to one or more embodiments of the invention;

FIG. 1B illustrates a side view of a reservoir with a frangible seal and holding a sterilized fluid for the lubrication of a surgical device according to one or more embodiments of the invention;

FIG. 2 illustrates a top perspective view of a reservoir with a frangible seal and holding a sterilized fluid for the lubrication of a surgical device according to one or more embodiments of the invention;

FIG. 3 illustrates a top perspective view of a reservoir with a frangible seal and holding a sterilized fluid for the lubrication of a surgical device deployed within a device for delivering a prosthetic implant according to one embodiment of the present invention;

FIG. 4 illustrates a top perspective view of a reservoir with a frangible seal and holding a sterilized fluid for the lubrication of a surgical device according to one or more embodiments of the invention;

FIG. 5 illustrates a top view of a reservoir with a frangible seal and holding a sterilized fluid for the lubrication of a surgical device according to one or more alternative embodiments of the invention;

FIG. 6 illustrates a flow diagram presenting a method for manufacturing a reservoir with a frangible seal and holding a sterilized fluid for the lubrication of a surgical device according to one or more embodiments of the invention;

FIG. 7A illustrates a top view of a reservoir with a frangible seal and holding a sterilized fluid for the lubrication of a surgical device system according to one or more embodiments of the invention;

FIG. 7B illustrates a perspective view of the reservoir of FIG. 7A;

FIG. 8A illustrates an end perspective view of a reservoir with a frangible seal and holding a sterilized fluid for the lubrication of a surgical device system according to one or more embodiments of the invention;

FIG. 8B illustrates a side perspective view of the reservoir of FIG. 8A;

FIG. 9A through 9D illustrate views of a reservoir with a frangible seal and holding a sterilized fluid in conjunction with a wicking material for the lubrication of a surgical device according to one or more embodiments of the invention; and

FIG. 9E through 9G illustrate views of a reservoir with a frangible seal and holding a sterilized fluid in conjunction with a wicking material for the lubrication of a surgical device during release and distribution of a fluid across a portion of the wicking material according to one or more embodiments of the invention;

### DETAILED DESCRIPTION OF CERTAIN EMBODIMENTS

By way of overview and introduction, embodiments of the invention are directed towards a fluid delivery system for assisting in activating lubricants on medical devices. More specifically the present application describes fluid delivery system that comprises a reservoir with a frangible seal and holding a sterilized fluid used to controllably apply such sterilized fluid to the surface of a medical device before or during use, for instance, to activate a surface coating on the medical device. Embodiments of the fluid delivery system present the reservoir with a frangible seal in conjunction with a wicking material that transmits the sterilized fluid to the entirety of the surface of the medial device.

By way of example and without limitation, the medical devices can include implantable devices (e.g., a pacemaker, breast implants, etc.) or any other device or surgical tool used during surgical procedures or that can be inserted into the human body during surgical procedures, either temporarily or permanently (e.g., a catheter, wire, broke, cannula, suction drain, etc.). Exemplary types of surface coatings can include, for example and without limitation, hydrophilic, hydrophobic, anti-microbial, or anti-thrombotic surface coatings, which are commonly applied to medical devices. It should be understood by those of skill in the art that there are many types of bioactive surface coatings currently available in the market, most notable among them being hydrophilic coatings, antimicrobial coatings, heparin coatings, lubricants, and the like.

By way of example and without limitation, the sterilized fluid inside the reservoir with a frangible seal can be used to activate the surface coating applied to a prosthetic implant delivery device as is shown and described in co-pending and commonly assigned U.S. patent application Ser. No. 15/352,079, entitled “DEVICE FOR THE DELIVERY OF A PROSTHETIC IMPLANT AND METHOD OF USE THEREOF,” filed on Nov. 15, 2016, which is hereby incorporated by reference as if set forth herein in its entirety. More specifically, the reservoir with a frangible seal can be used to selectively apply fluid to the interior surface of the prosthetic implant delivery device in a controlled manner. As such, application of the fluid activates a lubricious coating on the interior of the device that facilitates the delivery of a prosthetic implant through the delivery device.

In particular, embodiments of the present invention comprise a container of fluid or a “reservoir,” such as a packet, pouch, sachet, balloon or other receptacle for containing a fluid. The fluid can be sterile or non-sterile fluids or gels and can include, for example, physiologic saline or any other appropriate agent for activating a coating of a medical device. Preferably, the fluid delivery system also comprises a wicking material disposed in fluidic communication with a frangible seal on the reservoir and configured to controllably distribute the fluid released from the reservoir across the area of the wicking material. The wicking material can comprise one or more sheets of a biocompatible fabric or material. In addition, the reservoir can be attached to the wicking material by stapling, sewing, gluing or by placing the fluid within a reservoir formed by multiple layers of wicking material.

The reservoir preferably includes a frangible seal that can be selectively broken to permit the outflow of fluid from within the reservoir. For example, the walls of the reservoir can be sealed by any number of mechanisms and, preferably, one of the walls and/or seals, or a portion thereof, is frangible, meaning that excessive pressure generated within the reservoir by squeezing or twisting the reservoir may result in perforation of the frangible seal. Perforation of the frangible seal thereby allows for the release of the fluid directly onto the surgical device or, preferably, onto the biocompatible fabric, which in turn wets the device and consequently activates any coatings applied thereon.

For example, the fluid delivery system comprises a wicking material configured to be wettened with a fluid agent, such as sterile saline or water that, prior to use, is contained within in reservoir having a frangible seal. The reservoir is configured to rupture along the frangible seal upon application of a predetermined amount of mechanical pressure, for instance, by squeezing or twisting the assembly. Accordingly, prior to use, the fluid delivery system can be disposed within a medical device (catheter, wire, broke, cannula, suction drain, pacemaker, breast implant, etc.) and designed to bathe the device in the fluid when the reservoir is ruptured by wetting the wicking material and activating a coating on the medical device. Use of a wicking material to distribute the activating agent, e.g., saline, has benefits in terms of thoroughness of coating activation, ease of activation, less waste of material, time and surface area in the operating room or office.

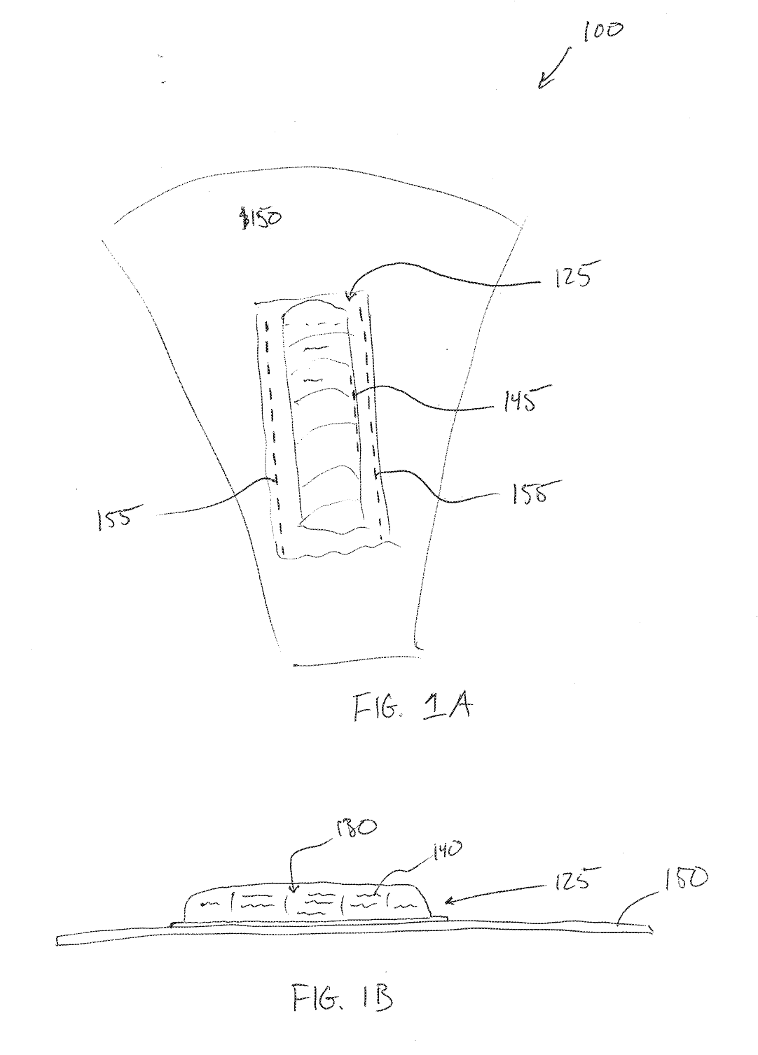

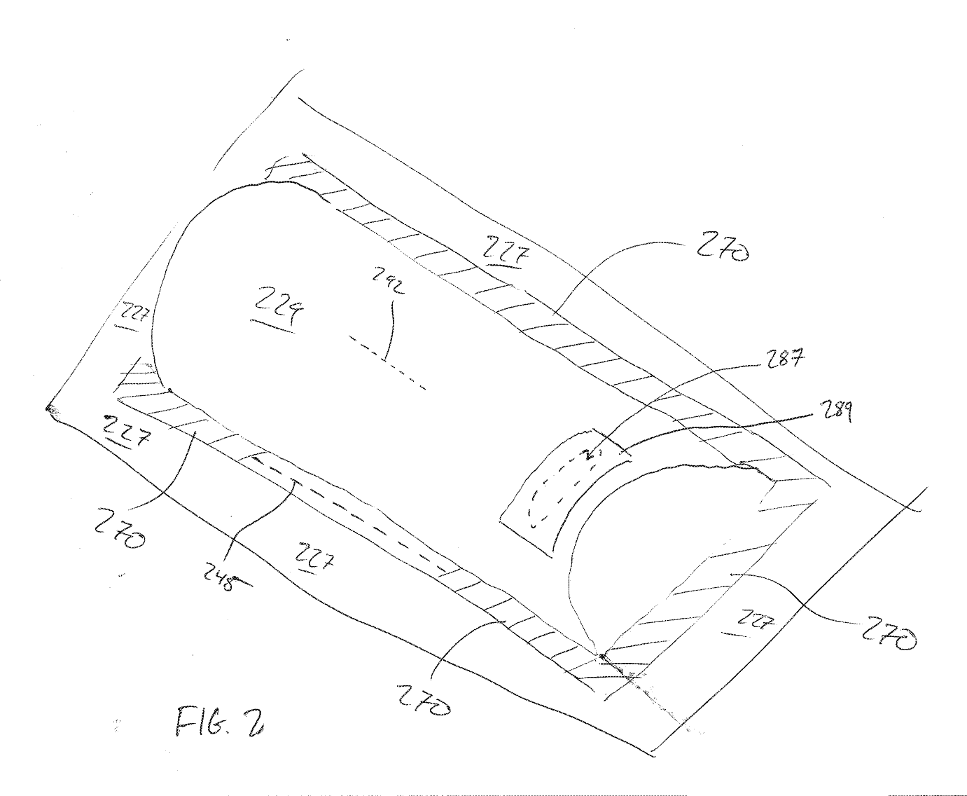

FIG. 1A is a high-level perspective view of an exemplary fluid delivery system 100 according to one or more embodiments of the invention. FIG. 1B illustrates a side-view of the device 100. As shown in FIG. 1A and FIG. 1B, the fluid delivery system 100 comprises a reservoir 125 that, in one or more exemplary embodiments, is affixed to a fluid distribution material 150.

The reservoir 125 comprises one or more walls enclosing an internal volume 130 suitable for containing a fixed amount of fluid 140 therein. By way of example and without limitation, the fluid contained within the reservoir can be a physiologic saline solution or other appropriate fluid. In some embodiments, the contents of the reservoir are sterile, which can be achieved by a number of available mechanisms known in the art. For instance, sterilization of the fluid contents can be performed prior to or after being sealed within the reservoir by autoclave using steam or a liquid cycle, the application of Ethylene Oxide gas, or gamma radiation, depending on the material used to fabricate the reservoir and the fluid contained therein. In one exemplary configuration, the reservoir 125 is formed of a non-permeable substrate comprising a polymer material selected from the set of polymers consisting of medical grade vinyl, medical grade PVC, medical grade nylon, mylar, and polyethylene.

The reservoir 125 can be constructed by arranging one or more sheets of substrate to provide exterior walls of the reservoir that surround an interior volume and then joining opposing and overlapping surfaces of the substrate layers together with one or more securements that extend along respective end margins of the substrate layers. The securement can be created by, for example and without limitation, bonding the one or more layers of the substrate together to form a hermetically sound seal between the walls of the substrate that is not permeable by the fluid contained therein. For instance, the walls of the reservoir 125 can be sealed by RF welding, heat sealing, pressure sealing, glue, adhesive tape, or other methods of sealing known to those of skill in the art. In some configurations the securement comprises a continuous bond extending along a length of the bonded margins of the substrate. Optionally, the securement comprises a series of individual bond sites intermittently spaced apart and arranged (e.g. linearly, evenly spaced, or both) along the length of the bonded margins of the substrate. Each such bond site can be formed by applying heat and pressure such that the overlapping sections of substrate adhere to one another.

The reservoir 125 also includes at least one outlet 145 that is configured to allow the out-flow of the fluid contained within the reservoir upon opening of the outlet 145. In some implementations, the reservoir can also be configured with a port to enable fluid to be injected into the reservoir during preparation of the fluid delivery system 100 (e.g., when filling the reservoir with the fluid) and, thereafter, closed to seal the fluid within the reservoir. It should be understood, however, that the reservoir can receive fluid via other inlets formed in the reservoir and then permanently sealed thereafter. In addition, as further described herein, the outlet 145 can be formed at other stages of preparation of the device 100, for instance, during manufacture of the reservoir and prior to filling the reservoir with fluid. In some implementations, the outlet can comprise a weakly sealed opening between two exterior walls of the reservoir. In addition, or alternatively, the exterior walls can comprise one or more dedicated openings formed in one or more of the exterior walls of the reservoir.

According to a salient aspect, the outlet 145 preferably comprises a frangible barrier, which is also referred to as a “temporary seal.” The frangible seal can be configured to be selectively broken by a user immediately prior to first use of the device and thereby provide one or more passageways between the interior volume of the reservoir and the exterior of the reservoir, thereby allowing for the outflow of fluid contained therein. For example, the frangible seal can be configured to be breached by external pressure upon the filled reservoir, e.g., by squeezing or twisting the reservoir.

Such a seal may be devised to be deliberately frangible by, for example and without limitation, forming a sufficiently thin or weak securement between two opposing layers of the substrate. Frangible seal technology is commonly used in the manufacture and implementation of modern medical and scientific devices, enabling the controlled release of testing reagents from respective sealed reservoirs; permitting premeasured mixing of pharmaceutical agents such as chemotherapeutic agents, cardiac medications, antibiotics, and many others. Frangible sealed reservoirs can use differential weld strengths that are designed to fail under specific pressures, allowing for a unit-of-use measure to be precisely delivered to a target well or reaction zone.

FIG. 2 illustrates a perspective view of a reservoir 225 constructed in accordance with one or more embodiments of the invention. As shown, the reservoir 225 can comprise a bottom wall 227 and an opposing top wall 229 that are overlapped so as to define an internal volume there between. In addition, FIG. 2 further illustrates opposing and abutting surfaces of the top and bottom walls permanently joined by a securement region 270 (shown in cross hatching) that extends continuously and almost completely around the internal reservoir. Preferably, the bond formed over the securement region 270 is “permanent” in that it is of such character that the bonded seam does not readily open.

Embodiments of the present invention comprise the use of multiple types and locations of frangible openings or seals, with one or more of the frangible openings described in FIG. 2 utilized in conjunction with a reservoir. As shown in FIG. 2, the bottom and top walls can be joined together along lengthwise section 245 with a securement/bond that is sufficiently thin or weak so as to be selectively broken by the user. By way of further example, a temporary seal in the reservoir 225 may be devised to be deliberately frangible by applying a seal over an opening/passageway provided through one or more walls of the reservoir with relatively a weak adhesive bond.

For example, as shown in FIG. 2, an opening or passageway 287 can be provided in a wall of the reservoir (e.g., wall 229) and the opening can be temporarily sealed by covering the passageway with a patch made, for example, of foil, and bonding the patch to the portion of the wall 289 that surrounds the passageway 287. By way of further example, as further shown in FIG. 2, a temporary seal 292 may be devised to be deliberately frangible by scoring or partially perforating an otherwise hermetically sound physical barrier, such as, wall 229. Other frangible seals can be provided, for example, the outlet can comprise one or more frangible joints having a prescribed rigidity and that are designed to be broken when a sufficient amount of force is applied to one or more sides of said frangible joints, thereby allowing the outflow of the fluid from the reservoir through voids created by the breaking of the frangible joints. The nature of the frangible seal determines the requisite amount of force required to break the seal.

Returning now to FIGS. 1A-1B, it should be understood by those of skill in the art that the reservoir 125 can include any number of outlets 145. The number of outlets and placement of each outlet(s) can also be defined so as to evacuate fluid 140 from within the reservoir 125 onto the wicking material 150, thereby allowing for distribution of the fluid in a controlled manner. It should also be understood by those of skill in the art that the size of the outlet 145 can also be defined to facilitate controlled evacuation of the fluid. For instance, a frangible outlet can be sufficiently small so as to allow the fluid to be released at a rate that facilitates optimal absorption and distribution of the fluid by the wicking material 150. By way of further example, the outlet 145 can be configured to allow the fluid to be evacuated at a rate that is a function of the external pressure applied by the user on the reservoir 125.

The fluid wicking material 150 is arranged to control the distribution of the fluid 140 upon release from the reservoir 125. Accordingly, the material is preferably in fluidic communication with one or more portions of the reservoir 125, or, at least, is positioned relative to the outlet 145, such that it can absorb the fluid as it exits from the reservoir.

In some embodiments, the wicking material 150 comprises a sheet of fabric material that is biocompatible. In addition, the wicking material 150 can be permanently or temporarily coupled to the reservoir 125, meaning that they can be held together either in a temporary or permanent fashion, which may comprise coupling in either a moveable or immovable fashion. The coupling can be provided by stapling, sewing, gluing, welding, etc. the wicking material 150 together with a portion of the reservoir 125. In addition, or alternatively, the reservoir 125 can be disposed within a pouch formed from multiple layers of wicking material joined or otherwise bonded together. For example, as shown in FIG. 1A, the wicking material 150 can be attached to the underside of the reservoir 125 at one or more points 155. As noted, use of a wicking material to absorb the activating agent (e.g., the fluid contained within the reservoir) has benefits in terms of the thoroughness of coating activation, ease of activation, limiting the waste of material (e.g., the amount of fluid, reservoir material and/or wicking material) and time and surface area use in the operating room or physician's office (e.g., the time and the space required to activate/prepare the medical device).

In one exemplary configuration, the wicking material 150 is formed of a substrate comprising a synthetic material, such as a polymer or a natural fabric. For instance, the wicking material 150 may comprise any woven or non-woven fabric material that suitably wicks fluid throughout its surface including, but not limited to lightweight stabilizer (60% polyester/40% Rayon), medium weight stabilizer (60% polyester/40% Rayon) and medium to heavyweight sew-in interfacing (100% Polyester). By way of further example, the substrate can comprise an engineered composite material such as the engineered composites having one or more of polypropylene, rayon, polyethylene terephthalate and acrylic fiber types sold under the brand name Stratex® Engineered Composites by Schweitzer-Mauduit International, Inc. of Alpharetta Ga.

According to a salient aspect, the material used to form the various components of the fluid delivery system are of the type that can be sterilized using various sterilization techniques commonly implemented in the medical and surgical fields. According to one embodiment, the reservoir is constructed from one or more sections of mylar, which is suitable for autoclave sterilization of the reservoir and its contents as the aluminum laminate resists degradation up to temperatures of 135 degrees Fahrenheit. Advantageously, the use of mylar allows for sterilization of the reservoir and its contents through the use of Ethylene Oxide gas as the foil is impermeable to gas.

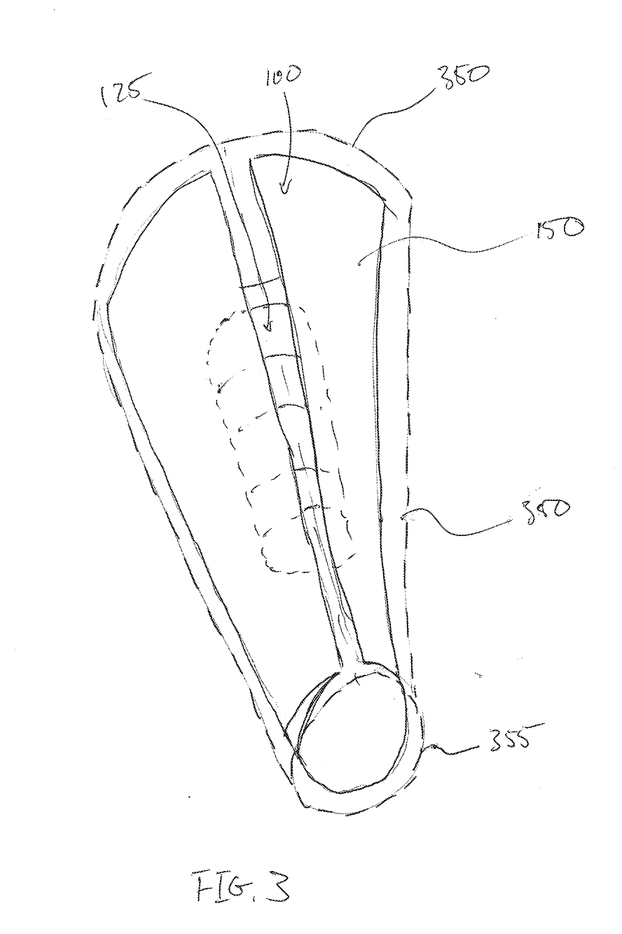

As a non-limiting practical example, the exemplary fluid delivery system 100 is further described as being used with a prosthetic implant delivery device shown in FIG. 3. FIG. 3 illustrates the fluid delivery system 100 (e.g., reservoir and wicking material) deployed within the interior volume of a generally cone shaped prosthetic implant delivery device 350, which is shown in phantom to provide a better view of the fluid delivery system 100 disposed of therein.

As shown in FIG. 3, the wicking material 150 can be folded around the reservoir 125 and the combination inserted within the interior of the implant delivery device 350. In this configuration, the wicking material 150 abuts a substantial portion of the inner surface of the implant delivery device 350, which is preferably coated with a lubricious coating that is activated upon contact with the activating agent contained within the reservoir 125. In use, a technician hydrates the wicking material by breaking the frangible seal on the reservoir, thereby allowing fluid to evacuate the reservoir for absorption by the wicking material. When hydrating, the wicking material 150 serves to wick the fluid throughout the interior of the implant delivery device 350, thereby activating a lubricious coating on its inner surface.

As shown in FIG. 3, the size and shape of the wicking material 150 is complementary to the size and shape of the internal surface of the medical device, e.g., 350. Accordingly, it can be appreciated that, depending on the application, the wicking material can be sized or shaped to correspond to the surface of the specific medical device to be lubricated. It can be further appreciated that the position of the wicking material 150 relative to the reservoir 125 or any frangible barriers/seals of the reservoir 125 can also be defined to facilitate uniform wetting of the wicking material and/or efficient operation (e g, minimal fluid waste). Continuing with the practical application shown in FIG. 3, after hydrating the wicking material 150, the technician can then pull the entire fluid delivery system 100 through the open end 355 of implant delivery device 350, to remove the device 100 and further facilitate distribution of the fluid across the entire surface of the implant delivery device 350 and activation of the coating previously deposited thereon.

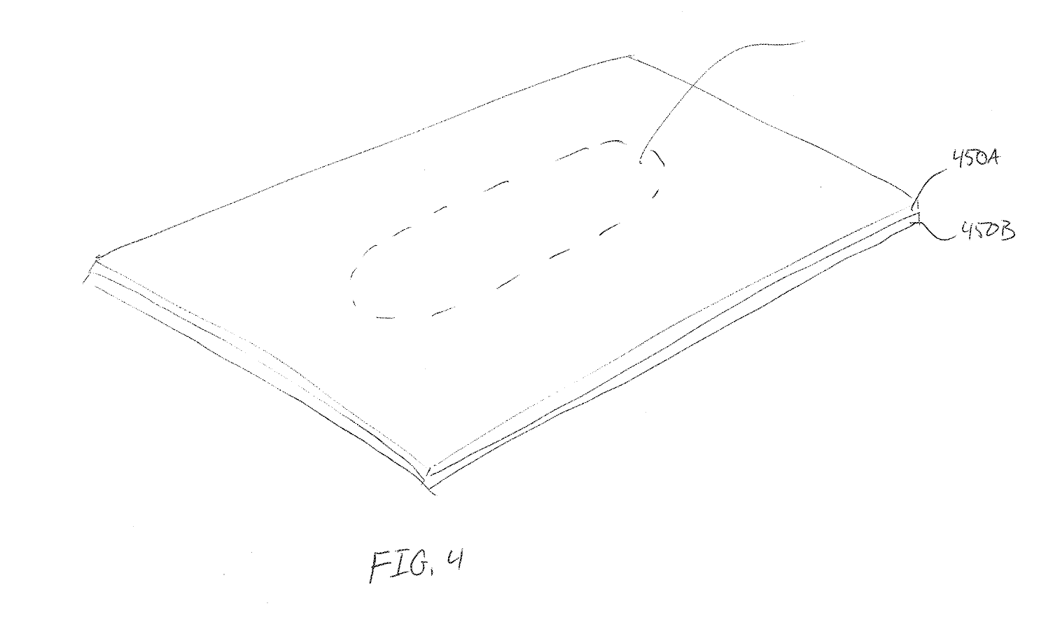

While the foregoing examples of the fluid delivery system 100 comprise a reservoir 125 placed on one side of the wicking material 150, other configurations can be implemented without departing from the scope of the invention described herein. For instance, in accordance with one or more embodiments, a fluid delivery system comprises a fluid-filled reservoir that is disposed between two layers (or sets of layers) of wicking material. For instance, FIG. 4 illustrates an exemplary fluid delivery system 400 in which the reservoir 425 is placed between and completely enclosed within two layers of wicking material 450A (top layer) and 450B (bottom layer). According to the embodiment of FIG. 4, the two layers of wicking material 450A and 450B can be joined together and/or joined to the pouch to maintain the position of the reservoir 425 there between.

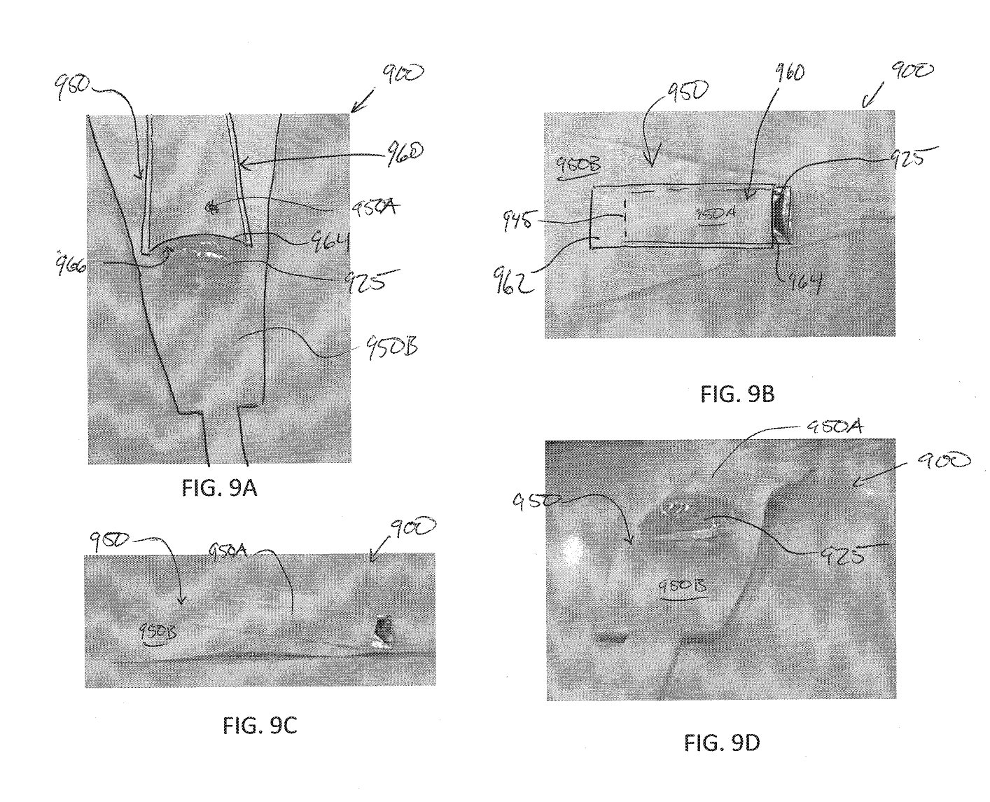

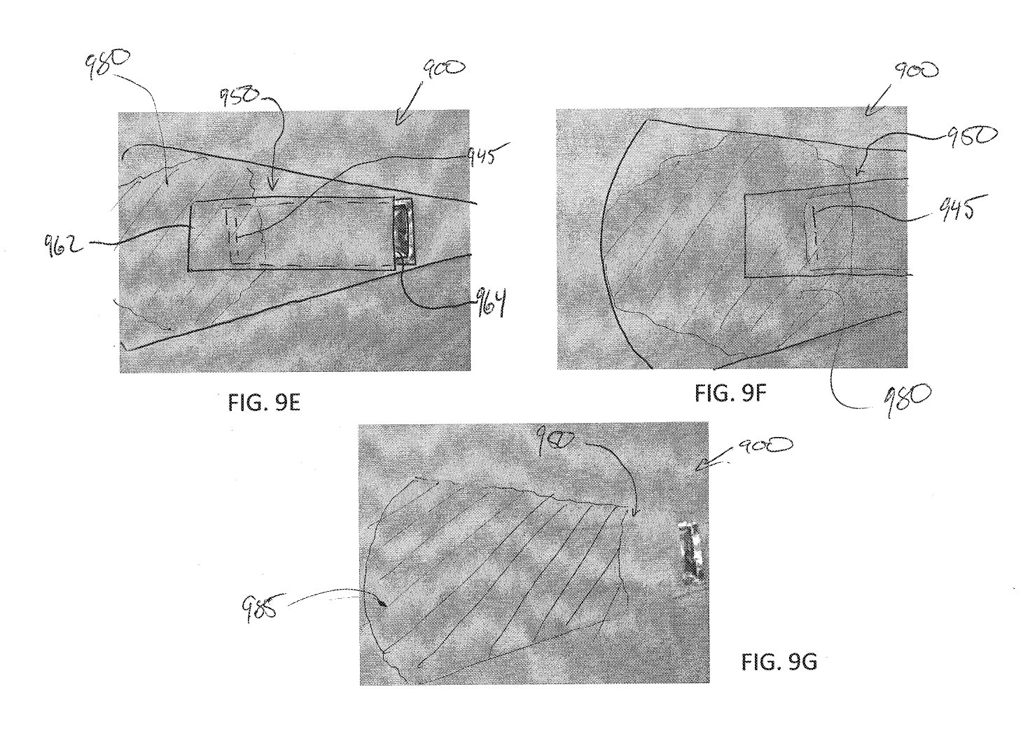

By way of further example, FIGS. 9A through 9D illustrate an exemplary fluid delivery system 900 in which the wicking material 950 comprises at least one bottom layer of material 950B and an opposing top layer of material 950A joined to a top surface of the bottom layer 950B to define a pouch 960 having a sealed first end 962, sealed sides and an open or otherwise unsealed second end 964, thereby forming an interior volume 966. The reservoir 925 containing fluid can be placed at least partially within the interior volume 966 of the pouch 960 and held in position without permanently attaching the reservoir 925 to the wicking material 950B and while allowing the reservoir to be inserted or removed as necessary. The layers of wicking material 950A and 950B can be joined together to define the pouch 960 by any of the previously-identified joining methods including, for example, ultrasound welding, radio frequency welding, heat sealing, gluing, sewing, etc.

FIGS. 9E through 9G illustrate various stages of absorption of the fluid that is released through a frangible seal 945 in the reservoir 925. As shown, in FIGS. 9B and 9E, for example, the frangible seal 945 is provided toward a first end of the reservoir 925 (obscured by top layer 950A with frangible seal 945 shown in dashed line) and positioned relative to the sealed first end 962 of the pouch 960. FIGS. 9E, 9F and FIG. 9G illustrate the progression of the distribution of fluid across the wicking material as a function of time and/or as a function of the amount of pressure placed on the reservoir 925. In particular, FIG. 9E and FIG. 9F, which illustrate a close up view of the fluid distribution device 900 shown in FIG. 9E. More specifically, FIG. 9E illustrates a stage in which fluid is absorbed and distributed across a portion 980 of the wicking material 950. FIG. 9G illustrates a subsequent stage, occurring later in time, and in which the absorbed fluid is distributed across a larger portion 985 of the wicking material 950.

The distribution of the fluid across the wicking material 950 and the saturation of the wicking material 950 can be controlled by design features in addition to the material properties of the substrate(s) that make up the wicking material. For instance, the amount of fluid that is released from the reservoir 925 through the frangible seal 945, and/or the rate at which fluid is released, can be controlled by the size and shape of the opening that the frangible seal defines. In addition, the frangible seal 945 can be designed such that the amount of fluid that is released from the reservoir 925 through the frangible seal 945, and/or the rate at which fluid is released, can be modulated by the amount of pressure the user places on the reservoir 925 during the process of breaking the frangible seal and thereafter. Moreover, the position of the one or more layers of wicking material relative to the frangible seal(s) 945 of the reservoir 925 can also be defined based on desired absorption characteristics. For example, a configuration in which at least the portion of the reservoir that is deliberately frangible is positioned between multiple layers of wicking material, e.g., as shown in FIG. 9A through 9G, can increase the surface area of the wicking material that is in fluidic communication with the frangible seal and, thus, facilitates efficient absorption of the fluid. A follow-on effect is that such a configuration serves to minimize the amount of fluid that runs off the wicking material prior to absorption.

By way of further example and without limitation, in accordance with one or more of the disclosed embodiments, a fluid-filled reservoir can be attached to the wicking material at one end thereof. For example, FIG. 5 illustrates a reservoir 525 having an interior volume 530 containing fluid 540 therein. The reservoir 525 can be attached to one end of the wicking material 550, e.g., along a seam 555 extending along one or more end margins of the reservoir, such that the wicking material is in fluidic communication with (e.g., disposed proximate to) the frangible seal 545, which can be selectively broken immediately prior to use. As described above, breaking the frangible 545 seal causes evacuation of the fluid 540 from within the reservoir 525 onto the wicking material 550 for distribution across the surface area of the wicking material 550.

In accordance with one or more embodiments of the invention, a fluid delivery system (and any medical device that the fluid delivery system is being used in conjunction with) can also be vacuum sealed within an outer bag. Such a configuration can have significance in accelerating the distribution of the fluid throughout the wicking material by drawing the fluid out of the reservoir and distributing it throughout the entirety of surface area of the wicking material, provided that the vacuum seal on the outer bag remains. It should also be understood by those of skill in the art that the fluid delivery system alone, or the combination of the fluid delivery system in conjunction with any medical device(s) for use therewith, can be vacuum sealed within a such a sterile pack to provide a sterilized assembly that can be activated and ready for use upon opening without requiring any further assembly of the fluid delivery system, the medical device, and/or the assembly comprising the foregoing components.

In accordance with one or more embodiments of the invention, the exemplary fluid delivery system can be manufactured according to an exemplary manufacturing routine 600 depicted in FIG. 6 and further described herein.

The routine 600 begins at step 605 which includes providing a reservoir having one or more walls that form an internal volume and that is suitable for receiving and containing a fluid therein. Preferably the reservoir has one or more openings suitable for filling the internal volume with the fluid. For instance, step 605 can comprise overlapping one or more sheets of non-permeable polymer substrate to form a reservoir-like container and permanently bonding one or more of the overlapped layers of polymer substrate to enclose the internal volume at least partially and leave one or more openings in the reservoir that can be used to fill the internal volume with fluid.

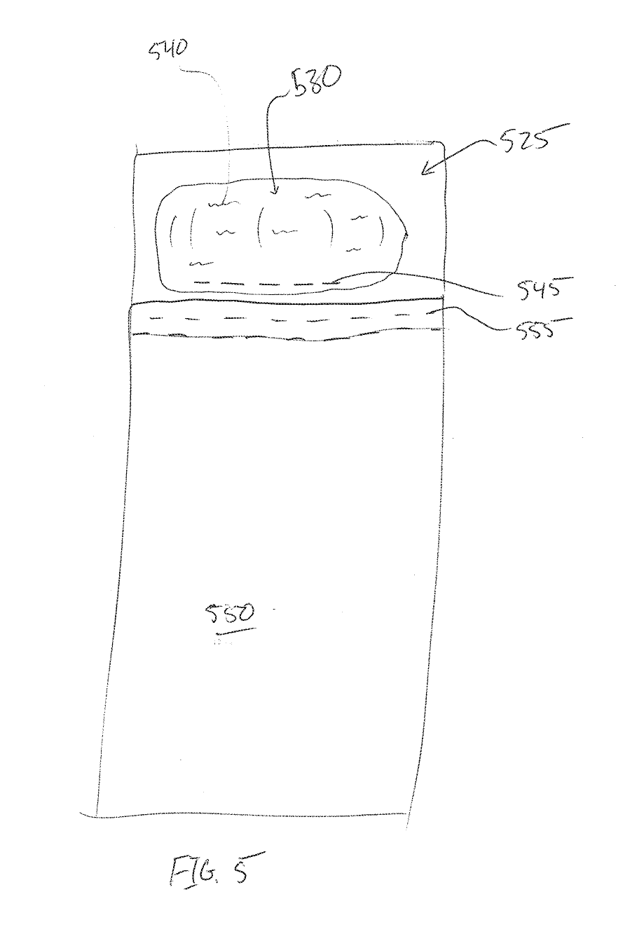

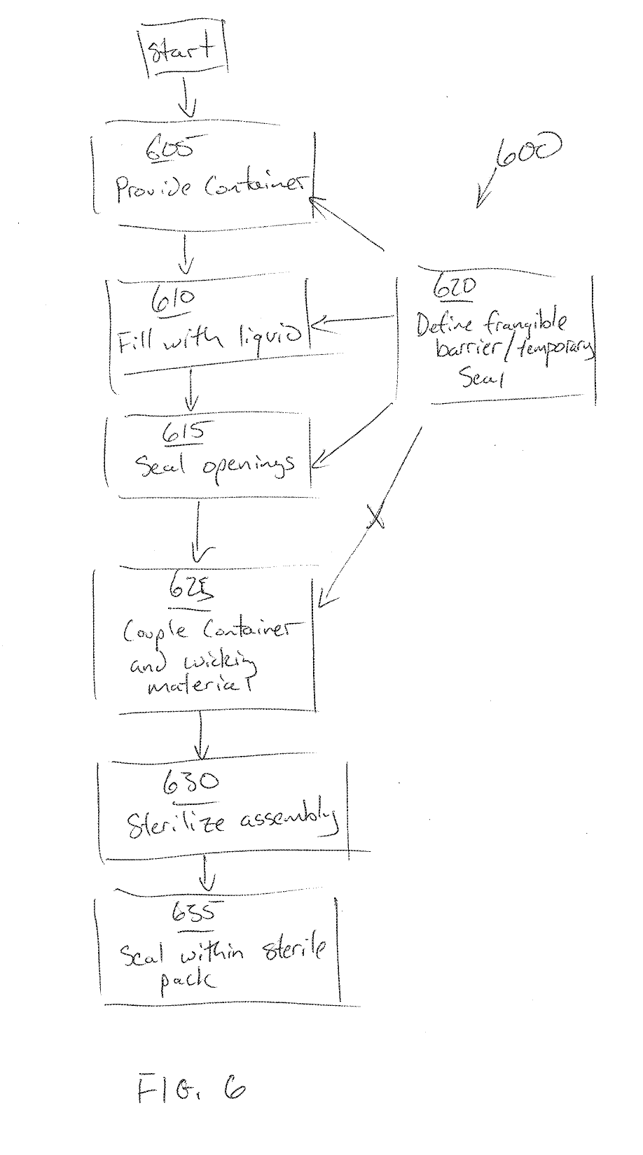

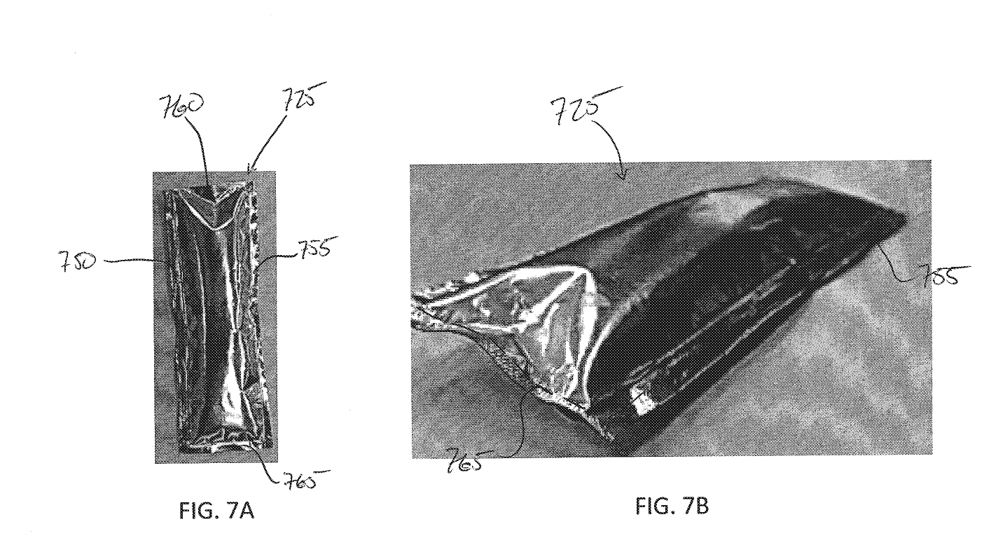

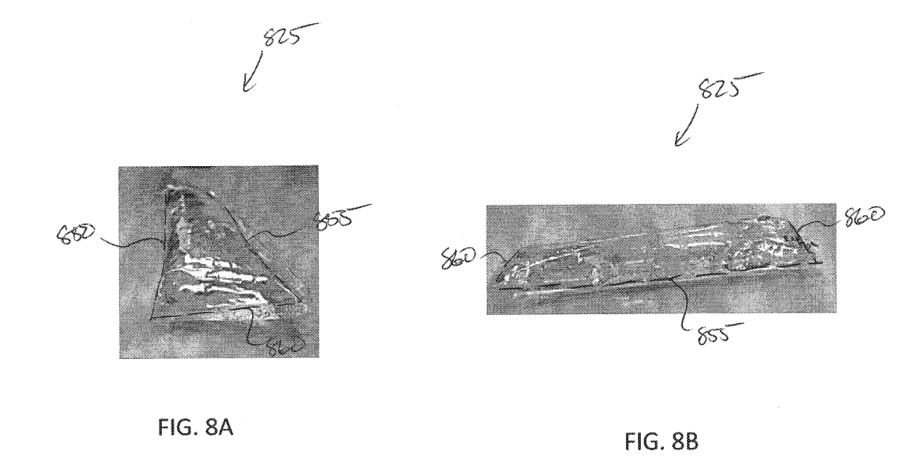

At step 610, the reservoir is filled with the fluid by, for example, injecting the fluid into the internal volume through the one or more openings. At step 615, the one or more openings of the reservoir are sealed to provide a fluid-filled, hermetically sealed, and non-permeable reservoir. FIGS. 7A and 7B provide a top-view and a perspective side view, respectively, of an exemplary fluid-filled reservoir 725 comprising two opposing sheets of polymer substrate arranged to define an internal volume therebetween. Two opposing side margins 750 and 755 and two opposing end margins 760 and 765 form a continuous seal around the perimeter of the reservoir 725. FIGS. 8A and 8B provide a top-view and a perspective side view, respectively, of an exemplary fluid-filled reservoir 825 comprising a sheet of substrate folded over along a left side edge 850 to provide two overlapping layers of from a single piece of substrate to define an internal volume therebetween. The open edges of the of the overlapping layers of substrate are sealed together along a right-side margin 855 and along two opposing end margins 860.

At step 620, a deliberately frangible barrier or “temporary seal” is formed on or in the reservoir. As noted, the seal can be devised to be deliberately frangible (e.g., operative to be broken or ruptured) upon application of a predetermined amount of mechanical pressure on the reservoir. Such mechanical pressure can take various forms, such as by squeezing or twisting the reservoir, the application of which allows for the outflow of fluid through the opening(s) provided by breaking/rupturing the frangible seal. In one or more embodiments, steps for providing a frangible seal 620 can be performed in connection with the step of sealing the reservoir, step 615. For example, at least one of the one or more openings used to fill the reservoir with fluid at step 610 can be sealed at step 615 with a temporary seal deliberately designed to be frangible.

Reservoirs comprising a frangible seal can utilize differential weld strengths that are designed to fail under specific pressures, allowing for a unit-of-use measure to be precisely delivered to a target well or reaction zone. More specifically, a frangible seal can be defined by forming a sufficiently thin or weak bond between two joined layers of substrate material. Whereas “permanent seals” between two layers of substrate can be created by a securement having a width or configuration that is not easily broken, e.g., a two (2) mm wide securement or two parallel one (1) mm wide securements, a frangible seal can be created by a securement that is relatively thinner, and thus weaker than the permanent securements. It should be understood by those of skill in the art that the permanent and temporary securements can be created using the same and/or different methods depending on the application and desired strength of the respective bonds.

By way of further example, a frangible seal can be formed by applying a patch over an opening/passageway that is provided through a wall of the reservoir using a relatively a weak adhesive bond between the surface of the patch and the opposing surface of the wall (e.g., the internal or external surface of the wall). By way of further example, a frangible seal can be formed by scoring or partially perforating an otherwise hermetically sound physical barrier, such as, the substrate defining the walls of the reservoir or a patch applied over a passageway.

In addition or alternatively, the exemplary steps for providing a frangible seal can be performed in connection with the steps for forming the reservoir, e.g., at step 605, or at some point subsequent to filling the container with fluid (step 610), and/or subsequent to performing the step of sealing the one or more openings used to fill the reservoir (step 620). For instance, step 605 can include scoring or partially perforating a portion of a sheet of substrate used to define a wall of the reservoir. By way of further example, step 605 can include the steps of cutting an opening or passageway through one of the layers of substrate used to define a wall of the reservoir and then applying a patch over the opening to provide a temporary or frangible seal.

At step 625, the reservoir is coupled to the wicking material. Various techniques can be used to couple or otherwise affix the reservoir to the wicking material including, but not limited to, stapling, sewing, gluing, placement of a pouch on or within one or more layers of the wicking material, etc. The combination of reservoir and wicking material as described herein provide the assembled fluid distribution system.

At step 630, the fluid distribution system comprising the fluid-filled reservoir and the wicking material are sterilized. Then, at step 635, the sterilized assembly is placed within a sterile pack that is then sealed. Alternatively, where the wicking material is already sterile, the reservoir is sterilized and then affixed to the wicking material for placement in the sterile pack for sealing. Optionally, routine 600 can comprise steps for arranging or combining the fluid distribution system with a medical device. The optional combining step can be performed prior to step 630 or step 635 and can also include positioning fluid distribution system relative to one or more surfaces of the medical device that are intended to be wetted using the system prior. The fluid distribution system and medical device may be placed in a sterile pack, which is then vacuum sealed.

FIGS. 1 through 9 are conceptual illustrations allowing for an explanation of the present invention. Those of skill in the art should understand that various aspects of the embodiments of the present invention could be implemented using different materials, fasteners, and minor design modifications. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention.

Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

Subject matter is described above with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. Those of skill in the art understand that other embodiments may be utilized and structural changes may be made without departing from the scope of the present invention. Likewise, a reasonably broad scope for claimed or covered subject matter is intended. The foregoing detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

While various embodiments of the present invention have been described above, it should be understood by those of skill in the art that such embodiments have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A fluid delivery system for use with a medical device, the fluid delivery system comprising:
a reservoir including:
one or more exterior walls arranged to define an internal volume and configured to contain a fluid therein, and
a frangible seal on a given one of the one or more exterior walls and configured to rupture in response to application of a pressure on one or more of the frangible seal and the one or more exterior walls, the frangible seal operative to provide an outlet for releasing the fluid contained within the reservoir; and
a wicking material coupled to the reservoir, wherein the wicking material is configured to absorb a volume of the fluid released from the reservoir and distribute the fluid across at least a portion of the wicking material.

2. The fluid delivery system of claim 1, wherein the wicking material comprises multiple layers of wicking material, wherein at least a top layer and a bottom layer are joined together to define a pouch there between and wherein the reservoir is positioned at least partially within the pouch such that the frangible seal is positioned to release the fluid from the reservoir into the pouch.

3. The fluid delivery system of claim 1, wherein the one or more exterior walls comprise one or more sheets of a polymer selected from the set of polymers consisting of medical grade vinyl, medical grade PVC, medical grade nylon, mylar, and polyethylene.

4. The fluid delivery system of claim 3, wherein the one or more sheets are overlaid to define the interior volume therebetween and wherein end margins of the one or more sheets are bonded together by one or more seals.

5. The fluid delivery system of claim 4, wherein the one or more seals include the frangible seal.

6. The fluid delivery system of claim 1, wherein the frangible seal comprises a temporary bond joining two overlapping end margins of the one or more exterior walls, wherein the temporary bond is sufficiently thin or weak such that it can be broken and cause the exterior walls to separate when the sufficient pressure is applied on the one or more exterior walls.

7. The fluid delivery system of claim 1, further comprising a patch applied over an opening in an exterior wall of the reservoir and secured to an interior or an exterior surface of the exterior wall.

8. The fluid delivery system of claim 1, wherein the frangible seal comprises a weakened section of an otherwise hermetically sound physical barrier between the internal volume and an exterior of the reservoir.

9. The fluid delivery system of claim 1, further comprising: an outer pouch disposed about the reservoir and the wicking material and enclosing the reservoir and the wicking material within the outer pouch.

10. The fluid delivery system of claim 9, wherein the outer pouch comprises a sealed outer pouch and wherein the reservoir and the wicking material are disposed within the outer pouch and under vacuum.

11. The fluid delivery system of claim 1, further comprising a medical device to which the fluid delivery system is to deliver the fluid.

12. A method of manufacturing a fluid delivery system for use with a medical device, the method comprising:
forming a reservoir with one or more exterior walls arranged to define an internal volume and configured to contain a fluid therein, wherein the reservoir includes one or more openings for receiving the fluid therein;
filling the internal volume of the reservoir with the fluid through the one or more openings;
sealing the one or more openings of the reservoir to provide a sealed and non-permeable container;
forming a frangible seal in one of the one or more exterior walls of the reservoir, wherein the frangible seal is configured to rupture in response to application of pressure on one or more of the frangible seal and the one or more exterior walls and provide an opening in the reservoir to release the fluid contained therein; and
coupling a wicking material to the reservoir, wherein the wicking material is configured to absorb the fluid released from the reservoir and distribute the fluid across at least a portion of the wicking material.

13. The method of claim 12, wherein forming the reservoir comprises forming a reservoir from one or more sheets of a polymer selected from the set of polymers consisting of medical grade vinyl, medical grade PVC, medical grade nylon, mylar, and polyethylene.

14. The method of claim 12, wherein coupling the wicking material comprises coupling a biocompatible fabric selected from the set of fabrics consisting of a lightweight stabilizer (60% polyester/40% Rayon), medium weight stabilizer (60% polyester/40% Rayon) and medium to heavyweight sew-in interfacing (100% Polyester).

15. The method of claim 12, wherein coupling the wicking material comprises one or more steps selected from the set of steps consisting of: stapling, sewing and gluing.

16. The method of claim 12, wherein coupling the wicking material comprises:
joining a top layer of wicking material to a bottom layer of wicking material to define a pouch having an open space for receiving the reservoir there between; and
placing the reservoir at least partially into the pouch defined by the top and bottom layers of wicking material.

17. The method of claim 12, wherein the step of forming the frangible seal comprises:
forming a temporary bond between two overlapping sections of the one or more sheets that is sufficiently thin or weak such that the temporary bond can be broken when pressure is applied on the one or more exterior walls.

18. The method of claim 12, comprising:
sterilizing the sealed and filled reservoir and wicking material attached thereto; and
sealing the assembly within a sterile pack.
